(19) United States
(12) Patent Application Publication
(10) Pub. No.: US 2022/0168194 A1
(43) Pub. Date: Jun. 2, 2022

(54) MOUSSE PRODUCTS, METHODS AND DEVICES

(71) Applicant: Honeywell International Inc., Morris Plains, NJ (US)

(72) Inventors: Yiu Keung LING, Charlotte, NC (US); Barry SETIAWAN, Charlotte, NC (US); Changming WANG, Charlotte, NC (US); Anthony ANZALONE, Charlotte, NC (US); Leonard STACHURA, Charlotte, NC (US)

(21) Appl. No.: 17/593,514

(22) PCT Filed: Mar. 20, 2020
(86) PCT No.: PCT/CN2020/080383

§ 371 Date: Sep. 20, 2021

Related U.S. Application Data
(60) Provisional application No. 62/822,505, filed on Mar. 22, 2019.

(51) Int. Cl. A61K 8/06; A61K 8/31; A61Q 5/00; A61Q 19/00

(52) U.S. Cl. CPC A61K 8/06; A61Q 19/00; A61Q 5/00; A61K 8/31

(57) ABSTRACT

A mousse composition having high gloss and good spreadability comprising: (i) carrier; (ii) one or more surface tension reducing agents that reduce the surface tension of the carrier; (iii) from greater than 5% to about 20% by weight, based on the weight of components (i)-(v), of foaming agent, wherein said foaming agent comprises at least about 30% by weight of trans-1234ze; and (iv) optionally one or more active components in addition to said surface tension reducing agents; and (v) optionally one or more adjuvants.

### CROSS REFERENCE TO RELATED APPLICATIONS

This application relates to and claims the priority benefit of U.S. Provisional application 62/822,505, filed Mar. 22, 2019, which is incorporated herein by reference in its entirety.

### FIELD OF THE INVENTION

This invention relates to mousse compositions, products and methods for delivery to the skin, hair and/or mucosa of animals, including particularly humans.

### BACKGROUND

Various aerosol and non-aerosol foams for the delivery of cosmetic, cleansing, and/or pharmaceutical components to the skin, hair and/or mucosa of animals are known in the prior art. In particular, formulations which upon dispensing produce a substantially homogeneous, expanding/expandable foam that breaks easily with shear are known in the art as “mousse” formulations or products.

In the formulation of foaming aerosol products, including mousses, an essential component is the propellant which is used to dispense the liquid concentrate from a container and onto the skin, hair and/or mucosa of the individual being treated. In manufacturing, the liquid concentrate portion of the formulation is typically first filled into the container, such as a can, which is sealed with a valve cup and then the propellant is introduced via pressure filling. It is also possible to prepare the container using under-the-cup filling where the propellant is filled and the valve cup sealed in one step.

One type of propellant that has been heretofore commonly used in the formulation of personal care products are compressed gases. Examples of compressed gases are nitrogen, carbon dioxide, etc., which remain as gases in the pressurized container. Depending on the type of liquid concentrate used, they can be partially solubilized in the liquid concentrate. The pressure that results from such a product can be controlled by the amount of propellant that is solubilized in the liquid concentrate and what is left in the head space. However, compressed gases are not widely used in personal care products since the pressure drops over the life of the can when it is being used resulting in different spray characteristics, lower spray rates and poorer foam quality over the useful life of the product. Such characteristics are considered generally undesirable from a consumer satisfaction standpoint.

Another type of propellant that has been heretofore commonly used in the formulation of personal care products are liquefied gas propellants or LGs. LGs are gases under normal temperature and pressure but become liquids under pressure above atmospheric. The advantage of using LGs is that, if properly designed, they can maintain a constant pressure throughout the life of the product in the can by converting from a liquid state to a gaseous state as the product in the can is dispensed for use by the consumer. This provides the user with a product that does not vary too much in its properties from one use to the next, which is desirable from a consumer satisfaction standpoint.

Certain hydrocarbons and hydrofluorocarbons, and dimethylether, have been commonly used as LGs. Fluorocarbons were the most widely used propellants up until the mid 1970's when their use was restricted because of concerns about damage to the ozone layer. Hydrocarbons have become the propellant of choice for many personal care applications because they are readily available and are less expensive than hydrofluorocarbons. Hydrocarbons used in the personal care industry include mixtures of propanes and butanes, most commonly two or more of isobutane, n-butane and propane. For example, the propellant known as “A-46” is a mixture of 15.15% by weight of propane and 84.85% by weight of isobutane. The designation A-46 reflects that the blend has a vapor pressure of 46 psig at 70° F.

One of the most commonly used hydrofluarocarbon propellants is HFC-152A, which has a vapor pressure of 62 psig at 70° F.

In the formulation of mousse products, applicants have come to appreciate that the choice and amount of propellant used can play a major role in the quality of the foam produced. Formulation parameters that applicants consider to be important to the success of a mousse product, and which are influence by the propellant which is used to dispense and form the mousse, include: (1) the initial stability of the foam dispensed, which represents the shape and surface properties of the foam; (2) the density of the foam which is produced, which influences the weight of the ingredients that are dispensed with each use; (3) the gloss of the foam; (4) the spreadability of the foam; and (5) the dispensing characteristics of the foam. In addition to the above; environmental concerns influence the success of a propellant for use in the formulation of mousse products since the propellant is generally released into the atmosphere as the product is used. Those skilled in the art appreciate that it therefore is a significant challenge to formulate new propellants for mousse products which are able to simultaneously satisfy these many important requirements of the foam.

Applicants have unexpectedly found that exceptionally high duality foams can be produced using a critical range of trans-1,3,3,3-tetrafluoropropene (trans-1234ze) as the propellant and/or the use of trans-1234ze in combination within specific amounts of the hydrocarbons propane and isobutane, as explained in detail hereinafter.

Applicants have also found that the use of certain surface reducing components in the mousse formulations, methods and devices enable the formation of the mousse foam at desired volume levels with unexpectedly low levels of the surface reducing components in the formulation. Additionally, it is possible to use these same surfactants to produce mousse formulation of unexpectedly high density, which has the desirably advantage of being able to deliver higher levels of cleansing surfactant to the user for a given dosage of the mousse foam.

### BRIEF DESCRIPTION OF THE DRAWINGS

FIG. C1A is a photograph of the resulting foam from Example C1A in the examples hereof.

FIG. C1B is a photograph of the resulting foam from Example C1B in the examples hereof.

FIG. C1C is a photograph of the resulting foam from Example C1C in the examples hereof.

FIG. C1D is a photograph of the resulting foam from Example C1D in the examples hereof.

FIG. C1E is a photograph of the resulting foam from Example C1E in the examples hereof.

FIG. C1F is a photograph of the resulting foam from Example C1F in the examples hereof.

FIG. 1A is a photograph of the resulting foam from Example 1A in the examples hereof.

FIG. 1B is a photograph of the resulting foam from Example 1B in the examples hereof.

FIG. 10 is a photograph of the resulting foam from Example 10 in the examples hereof.

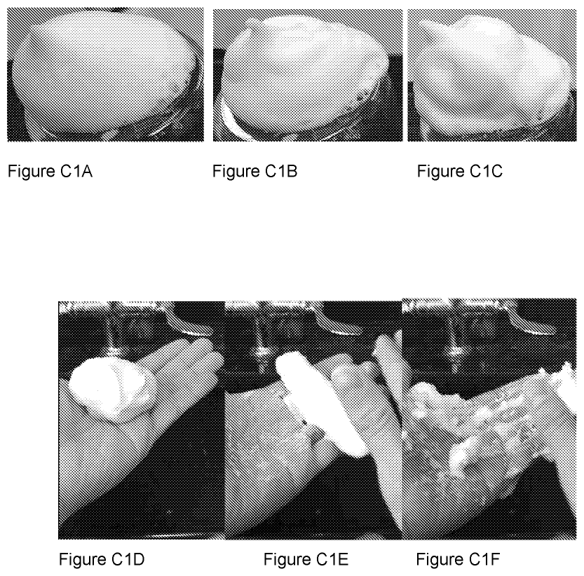

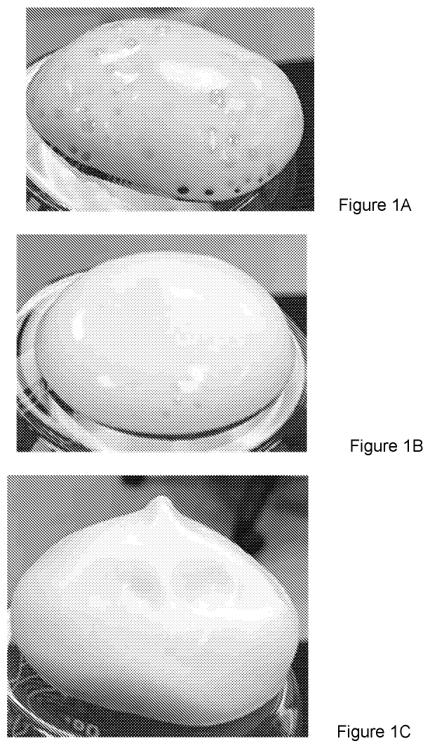

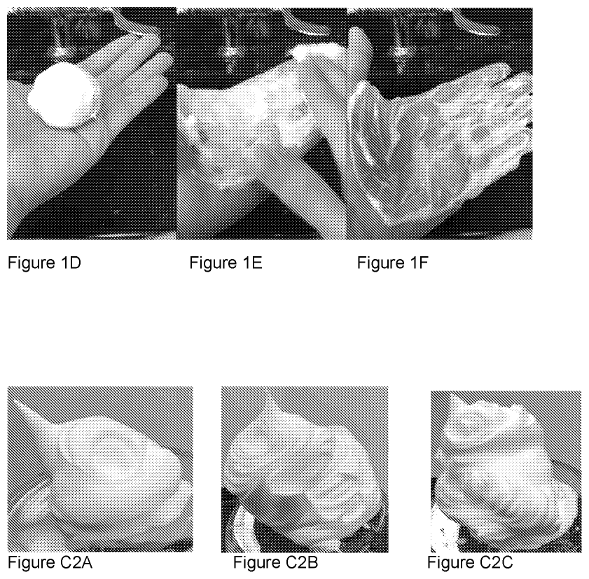

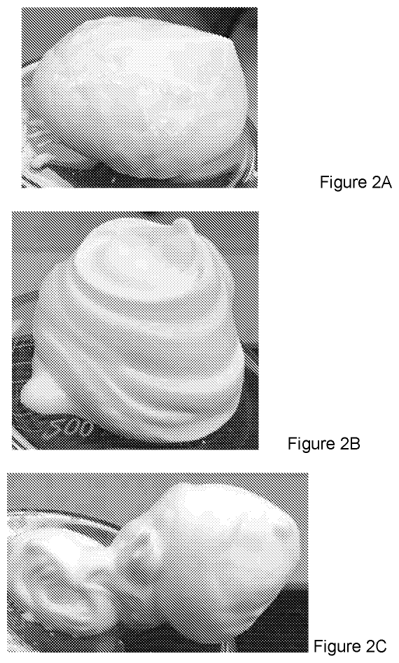

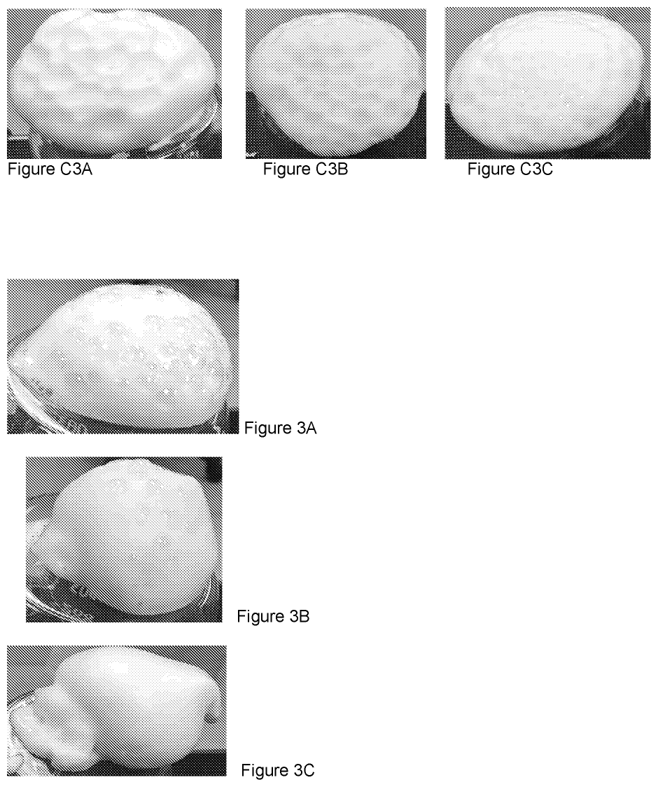

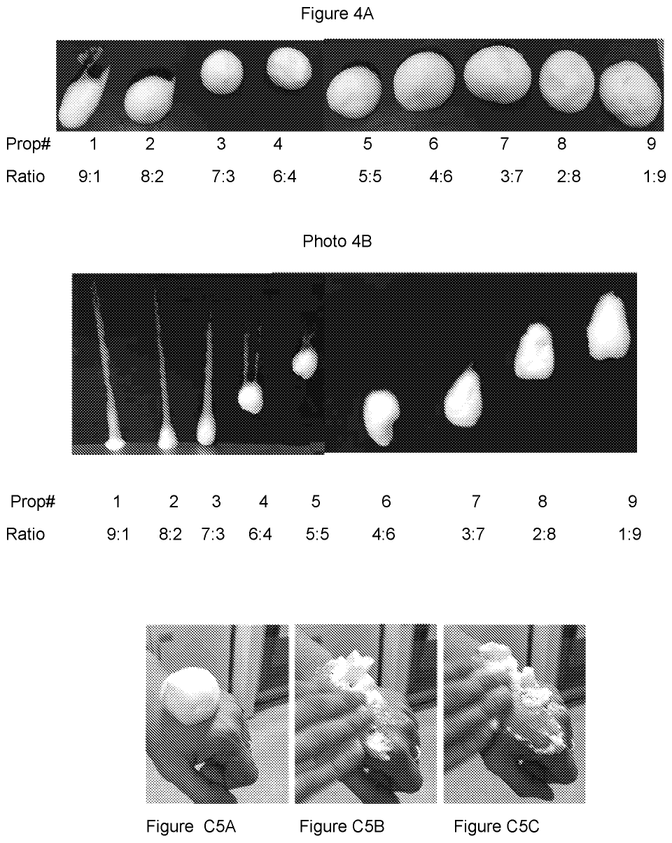

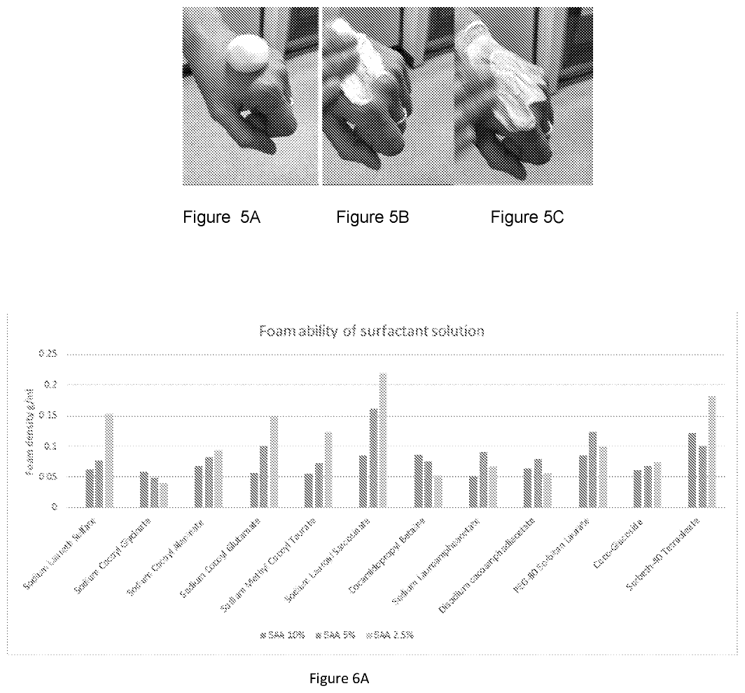

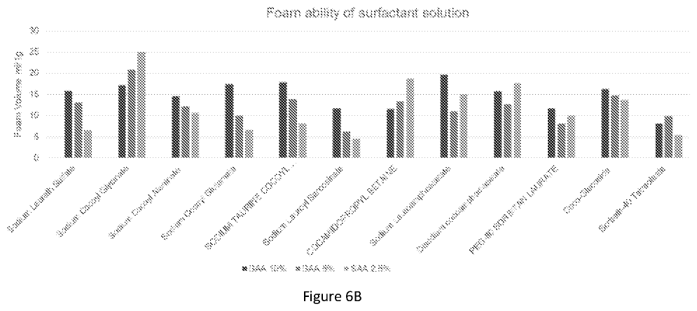

FIGS. 1D, 1E and 1F are photographs of the resulting foam from Example 1C in the examples hereof.

FIG. C2A is a photograph of the resulting foam from Example C2A in the examples hereof.

FIG. C2B is a photograph of the resulting foam from Example C2B in the examples hereof.

FIG. C2C is a photograph of the resulting foam from Example C2C in the examples hereof.

FIG. 2A is a photograph of the resulting foam from Example 2A in the examples hereof.

FIG. 2B is a photograph of the resulting foam from Example 2B in the examples hereof.

FIG. 2C is a photograph of the resulting foam from Example 2C in the examples hereof.

FIG. C3A is a photograph of the resulting foam from Example C3A in the examples hereof.

FIG. C3B is a photograph of the resulting foam from Example C3B in the examples hereof.

FIG. C3C is a photograph of the resulting foam from Example C3C in the examples hereof.

FIG. 3A is a photograph of the resulting foam from Example 3A in the examples hereof.

FIG. 3B is a photograph of the resulting foam from Example 3B in the examples hereof.

FIG. 3C is a photograph of the resulting foam from Example 3C in the examples hereof.

FIG. 4A is a photograph of the resulting foam from Example 4 in the examples hereof.

FIG. 4B is a photograph of the resulting foam from Example 4 in the examples hereof.

FIG. C5A is a photograph of the resulting foam from Example C5A in the examples hereof.

FIG. C5B is a photograph of the resulting foam from Example C5B in the examples hereof.

FIG. C5C is a photograph of the resulting foam from Example C5C in the examples hereof.

FIG. 5A is a photograph of the resulting foam from Example 5A in the examples hereof.

FIG. 5B is a photograph of the resulting foam from Example 5B in the examples hereof.

FIG. 5C is a photograph of the resulting foam from Example 5C in the examples hereof.

FIG. 6A is a chart of foam density data from Example 6.

FIG. 6B is a chart of the foam volume data from Example 6.

### SUMMARY OF THE INVENTION

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze; and

(iv) optionally one or more active components in addition to said surface tension reducing agents; and

(v) optionally one or more adjuvants. For the purposes of convenience, mousse compositions according to this paragraph are sometimes referred to herein as Mousse 1.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents;

and (v) optionally one or more adjuvants. For the purposes of convenience, mousse compositions according to this paragraph are sometimes referred to herein as Mousse 2.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 3.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 6% to about 20% by weight, based on the weight of components (i)-(v) of one or more surface tension reducing agents that reduce the surface tension of the carrier;

(iii) from about 7.5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, mousse compositions according to the paragraph are sometimes referred to herein as Mousse 4.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, mousse compositions according to this paragraph are sometimes referred to herein as Mousse 5.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a glossiness of 4 or greater. For the purposes of convenience, mousse compositions according to this paragraph are sometimes referred to herein as Mousse 6.

The present invention includes mousse compositions having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, mousse compositions according to this paragraph are sometimes referred to herein as Mousse 7.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants, wherein said foam has a spreadability of 4 or greater, a glossiness of 4 or greater and a initial foam stability of 4 or greater. For the purposes of convenience, mousse compositions according to this paragraph are sometimes referred to herein as Mousse 8.

The present invention includes mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 9.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 10.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 11.

The present invention includes mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, mousse compositions according to this paragraph are sometimes referred to herein as Mousse 12.

The present invention includes mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a glossiness of 4 or greater. For the purposes of convenience, mousse compositions according to this paragraph are sometimes referred to herein as Mousse 13.

The present invention mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, mousse compositions according to this paragraph are sometimes referred to herein as Mousse 14.

The present invention includes mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants, wherein said foam has a spreadability of 4 or greater, a glossiness of 4 or greater and a initial foam stability of 4 or greater. For the purposes of convenience, mousse compositions according to this paragraph are sometimes referred to herein as Mousse 15.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 16.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 17.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 18.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, mousse compositions according to this paragraph are sometimes referred to herein as Mousse 19.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a glossiness of 4 or greater. For the purposes of convenience, mousse compositions according to this paragraph are sometimes referred to herein as Mousse 20.

The present invention includes mousse compositions having high gloss and good spreadability on a user comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, mousse compositions according to this paragraph are sometimes referred to herein as Mousse 21.

The present invention includes mousse compositions having high gloss and good spreadability on a user comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier, wherein said surface tension reducing agent is selected from the group consisting of sodium cocoyl glycinate, cocoamidopropyl betaine, sorbeth-40 tetraoleate and disodium cocaamphodiacetate;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 22.

The present invention includes mousse compositions having high gloss and good spreadability on a user comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier, wherein said surface tension reducing agent is selected from the group consisting of sodium cocoyl glycinate, cocamidopropyl betaine, sorbeth-40 tetraoleate and disodium cocaamphodiacetate;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, mousse compositions according to this paragraph are sometimes referred to herein as Mousse 23.

The present invention also includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 1.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 2.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 3.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 6% to about 20% by weight, based on the weight of components (i)-(v) of one or more surface tension reducing agents that reduce the surface tension of the carrier;

(iii) from about 7.5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 4.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 5.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a glossiness of 4 or greater. For the purposes of convenience, methods according to this paragraph are sometimes referred to herein as Method 6.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a spreadability of 4 or greater, a glossiness of 4 or greater, an initial foam stability of 3 or greater. For the purposes of convenience, methods according to this paragraph are sometimes referred to herein as Method 7.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants; and

(b) applying said mousse formulation to the user, wherein said foam has a spreadability of 4 or greater, a glossiness of 4 or greater and a initial foam stability of 4 or greater. For the purposes of convenience, methods according to this paragraph are sometimes referred to herein as Method 8.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 9.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 10.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 11.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 12.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a glossiness of 4 or greater. For the purposes of convenience, methods according to this paragraph are sometimes referred to herein as Method 13.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a spreadability of 4 or greater, a glossiness of 4 or greater, an initial foam stability of 3 or greater. For the purposes of convenience, methods according to this paragraph are sometimes referred to herein as Method 14.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants; and

(b) applying said mousse formulation to the user, wherein said foam has a spreadability of 4 or greater, a glossiness of 4 or greater and a initial foam stability of 4 or greater. For the purposes of convenience, methods according to this paragraph are sometimes referred to herein as Method 15.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 16.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 17.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 18.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants; and

(b) applying said mousse formulation to the user. For the purposes of convenience, methods according to this paragraph are sometimes referred to herein as Method 19.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a glossiness of 4 or greater. For the purposes of convenience, methods according to this paragraph are sometimes referred to herein as Method 20.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user, wherein said foam has a spreadability of 4 or greater, a glossiness of 4 or greater, an initial foam stability of 3 or greater. For the purposes of convenience, methods according to this paragraph are sometimes referred to herein as Method 21.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier, wherein the surface tension reducing agent is selected from the group consisting of sodium cocoyl glycinate, cocoamidopropyl betaine, sorbeth-40 tetraoleate and disodium cocoamphodiacetate;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants; and

(b) applying said mousse formulation to the user to form a mousse foam, preferably to form a mousse foam having a density of about 0.05 g/ml or less. For the purposes of convenience, methods according to this paragraph are sometimes referred to herein as Method 22.

The present invention includes methods of forming a mousse having high gloss and good spreadability on a user comprising:

(a) providing a mousse formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier, wherein the surface tension reducing agent is selected from the group consisting of sodium cocoyl glycinate, cocamidopropyl betaine, sorbeth-40 tetraoleate and disodium cocaamphodiacetate;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants; and

(b) applying said mousse formulation to the user to the user to form a mousse foam, preferably to form a mousse foam having a density of about 0.05 g/ml or less. For the purposes of convenience, methods according to this paragraph are sometimes referred to herein as Method 23.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze; and

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 1.

The present invention also includes articles of manufacture that dispense a mousse composition having high gloss and good spreadability on a user, said article comprising:

(a) a container containing a formulation under pressure and being openable to dispense said formulation, said formulation comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents;

and (v) optionally one or more adjuvants. For the purposes of convenience, articles of manufacture according to this paragraph are sometimes referred to herein as Mousse Dispenser 2.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 3.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 6% to about 20% by weight, based on the weight of components (i)-(v) of one or more surface tension reducing agents that reduce the surface tension of the carrier;

(iii) from about 7.5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 4.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 5.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a glossiness of 4 or greater. For the purposes of convenience, articles of manufacture according to this paragraph are sometimes referred to herein as Mousse Dispenser 6.

The present invention also includes articles of manufacture that dispense a mousse composition having high gloss and good spreadability on a user, said article comprising:

(a) a container containing a formulation under pressure and being openable to dispense said formulation, said formulation comprising:

(a) providing a mousse formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, articles of manufacture according to this paragraph are sometimes referred to herein as Mousse Dispenser 7.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants, wherein said foam has a spreadability of 4 or greater, a glossiness of 4 or greater and an initial foam stability of 4 or greater. For the purposes of convenience, articles of manufacture according to this paragraph are sometimes referred to herein as Mousse Dispenser 8.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 9.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 10.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 11.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 12.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a glossiness of 4 or greater. For the purposes of convenience, articles of manufacture according to this paragraph are sometimes referred to herein as Mousse Dispenser 13.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, articles of manufacture according to this paragraph are sometimes referred to herein as Mousse Dispenser 14.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v) of one or more surface tension reducing agents that reduce the surface tension of the carrier;

(iii) from greater than 5% to about 20% by weight, based on the weight of components (i)-(v), of foaming agent, wherein said foaming agent from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants, wherein said foam has a spreadability of 4 or greater, a glossiness of 4 or greater and a initial foam stability of 4 or greater. For the purposes of convenience, articles of manufacture positions according to this paragraph are sometimes referred to herein as Mousse Dispenser 15.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 16.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 17.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 15% by weight, based on the weight of components (i)-(v), of foaming agent, wherein said foaming agent comprises at least about 50% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 18.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises from about 30% to about 70% by weight of trans-1234ze, from about 4% to about 11% by weight of propane and from about 25% to about 60% by weight of isobutane;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 19.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and (v) optionally one or more adjuvants, wherein said foam has a glossiness of 4 or greater. For the purposes of convenience, articles of manufacture according to this paragraph are sometimes referred to herein as Mousse Dispenser 20.

The present invention also includes articles of manufacture that dispense a mousse composition having high gloss and good spreadability on a user, said article comprising:

(i) from about 50% by weight to about 90% by weight, based on the weight of components (i)-(v), of carrier;

(ii) from about 0.5% to about 20% by weight, based on the weight of components (i)-(v), of one or more surface tension modifiers to reduce the surface tension of said carrier, provided that if said surface tension modifier is present in an amount of less than about 3% by weight, then said component (v) is present and comprises an adjuvant which aids in the formation of foam cell walls from said carrier;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants, wherein said foam has a spreadability of 4 or greater, a glossiness of 4 or greater, an initial foam stability of 3 or greater. For the purposes of convenience, articles of manufacture according to this paragraph are sometimes referred to herein as Mousse Dispenser 21.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier;

(ii) from about 3% to about 20% by weight, based on the weight of components (i)-(v), of one or more surface tension reducing agents that reduce the surface tension of the carrier, wherein said surface tension reducing agent is selected from the group consisting of sodium cocoyl glycinate, cocamidopropyl betaine, sorbeth-40 tetraoleate and disodium cocaamphodiacetate;

(iii) from greater than 5% to about 20% by weight, based on the weight of components (i)-(v), of foaming agent, wherein said foaming agent comprises at least about 30% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 22.

The present invention also includes articles of manufacture that dispense a mousse composition having high gloss and good spreadability on a user, said article comprising:

(a) a container containing a formulation under pressure and being openable to dispense said formulation, said formulation comprising:

(i) from about 50% by weight to about 90% by weight, based on the weight of components (i)-(v), of carrier, wherein said carrier comprises at least 90% by weight of water;

(ii) from about 3% to about 20% by weight, based on the weight of components (i)-(v) of one or more surface tension reducing agents that reduce the surface tension of the carrier, wherein said surface tension reducing agent is selected from the group consisting of sodium cocoyl glycinate, cocamidopropyl betaine, sorbeth-40 tetraoleate and disodium cocaamphodiacetate;

(iii) from greater than 5% to about 15% by weight, based on the weight of components (i)-(v), of foaming agent, wherein said foaming agent comprises at least about 40% by weight of trans-1234ze;

(iv) optionally one or more active components in addition to said surface tension reducing agents; and

(v) optionally one or more adjuvants. For the purposes of convenience, articles of manufacture according to this paragraph are sometimes referred to herein as Mousse Dispenser 23.

### DETAILED DESCRIPTION

The present invention provides mousse compositions comprising (i) carrier; (ii) at least one surface tension modifier, (iii) foaming agent, (iv) optionally one or more active components in addition to surface tension modifiers, and (v) optionally one or more adjuvants. For the purposes of convenience, components (i), (ii), (iv) and (v) together are sometimes referred to herein as the “concentrate” portion of the mousse composition. As those skilled in the art will understand, the particular amount and type of components used in the concentrate portion of the mousse of the present invention, including each of Mousse 1-23, can be varied and adjusted to achieve the particular target uses of the product within the objects and teachings of the present invention.

Uses

Broadly speaking, the mousse compositions of the present invention can be used to deliver to the skin, and/or the hair, and/or a mucous membrane of an animal, preferably a human, one or more components for affecting the appearance, the condition or the health of the user to whom it is applied. Such uses include, but are not limited to, use as personal care products. As used herein, the term “personal care” refers to the wide variety of products that as applied affect the health and/or appearance of the user. As such, the term “personal care” includes within its meaning both cosmetic uses and medicinal/drug uses. As used herein, the term “cosmetic uses,” “cosmetic products” and similar terms refer to uses, products and the like which have an impact on the appearance and/or feel and/or odor and/or cleanliness of the user. As used herein, the term “medicinal” and “drug” uses and products refers to uses, products and the like which impact the health of the user by improving or treating an illness, injury or other health condition of the user or as a protectant for skin or hair of the user.

The present invention therefore includes use of a mousse of the present invention, including each of Mousse 1-23, as a personal care product.

In general, the personal care use and products of the present invention can be classified as “rinse-off” products or “leave-on” products. As used herein, the term “rinse-off” product refers to a product which is intended to be applied, rubbed in or onto the skin or hair of the user, and then rinsed-off, typically with water. In contrast, a “leave-on” product refers to a product which is intended to be applied, optionally rubbed into the skin or hair of the user, and then left on the body for an extended period of time, preferably to have a relatively longer term impact on the appearance and/or feel and/or odor and/or health of the user.

The present invention therefore includes use of a mousse of the present invention, including each of Mousse 1-23, as a rinse-off personal care product.

The present invention therefore includes use of a mousse of the present invention, including each of Mousse 1-23, as a leave-on personal care product.

The present invention therefore also includes use of a mousse of the present invention, including each of Mousse 1-23, as a cosmetic.

The present invention therefore also includes use of a mousse of the present invention, including each of Mousse 1-23, as a rinse-off cosmetic.

The present invention therefore also includes use of a mousse of the present invention, including each of Mousse 1-23, as a leave-on cosmetic.

The present invention therefore also includes use of a mousse of the present invention, including each of Mousse 1-23, as a rinse-off medicinal/drug product.

The present invention therefore also includes use of a mousse of the present invention, including each of Mousse 1-23, as a leave-on medicinal/drug product.

Examples of cosmetics uses and cosmetic products include shampoos, conditioners, skin cleaners, facial cleaners, hair styling, shaping and coloring products, shave creams, sun tan products, sun block products, skin moisturizers, perfumes, facial makeup preparations and deodorants.

The present invention therefore also includes use of a mousse of the present invention, including each of Mousse 1-23, as a shampoo.

The present invention therefore also includes use of a mousse of the present invention, including each of Mousse 1-23, as a hand wash.

The present invention therefore also includes use of a mousse of the present invention, including each of Mousse 1-23, as a body wash.

The present invention therefore also includes use of a mousse of the present invention, including each of Mousse 1-23, as a facial cleanser.

The present invention therefore also includes use of a mousse of the present invention, including each of Mousse 1-23, as a shave cream. The present invention therefore also includes use of a mousse of the present invention, including each of Mousse 1-22, as a skin moisturizer.

The present invention therefore also includes use of a mousse of the present invention, including each of Mousse 1-23, as a sun screen.

The present invention therefore also includes use of a mousse of the present invention, including each of Mousse 1-22, as a leave-on facial mousse.

The present invention therefore also includes use of a mousse of the present invention, including each of Mousse 1-23, as a hair coloring mousse.

The present invention therefore also includes use of a mousse of the present invention, including each of Mousse 1-23, as a hair styling mousse.

The present invention therefore also includes use of a mousse of the present invention, including each of Mousse 1-23, as a hair conditioning mousse.

Examples of medicinal/drug uses and medicinal/drug products include topical treatments for skin or hair problems or diseases (such as dandruff or skin rashes), systemic drugs or medicines applied transdermally, and products for treatment or inhibition of the biological conditions of the user (such as antiperspirants and sunscreens)

It will be appreciated by those skilled in the art that many personal products will fall within the category of both cosmetics and medicinal/drug products, and all such dual use products and uses are within the scope of the present invention. For example, a shampoo is a cosmetic because its intended use is to cleanse the hair, and an antidandruff treatment is a medicinal/drug because its intended use is to treat dandruff. Consequently, an anti-dandruff shampoo falls within the category of both cosmetic and medicinal/drug because it cleanses the hair and treats dandruff. Among other personal care products that are both cosmetic and medicinal/drug categories include are toothpastes that contain fluoride, deodorants that are also antiperspirants, and moisturizers and makeup that have sun-protection components.

Mousse

Carrier

The mousse of the present invention, including each of Mousse 1-23, comprises a carrier that can comprise an aqueous carrier, a non-aqueous carrier or a combination of aqueous and non-aqueous carriers. For example, the carrier of the mousse, including each of Mousse 1-23, may comprise water forming a continuous phase with one or more of, or all of, the other components of the concentrate dissolved, suspended, dispersed and/or emulsified therein. For example, in the case of a hair conditioning shampoo product the carrier may comprise water in which particles of water-insoluble conditioning agent are dispersed or suspended, along with other active components and adjuvants that may be present. In the case of a sunscreen mousse products by way of further example, the carrier may comprise water in which oil soluble sunscreen actives are emulsified. The water in preferred embodiments is preferably distilled and/or deionized.

The water in preferred embodiments is preferably present in the mousse, including each of Mousse 1-23, in amount of from about 30% to about 98% by weight.

The water in preferred embodiments is preferably present in the mousse, including each of Mousse 1-23, in amount of from about 50% to about 98% by weight.

The water in preferred embodiments is preferably present in the mousse, including each of Mousse 1-23, in amount of from about 60% to about 98% by weight.

In preferred embodiments, the mousse, including each of Mousse 1-23, comprises a carrier that comprises, or consists essentially of, or consists of water.

The present mousse, including each of Mousse 1-23, may comprise a carrier that is an organic carrier forming a continuous phase with one or more of, or all of, the other components of the concentrate dissolved, suspended, dispersed and/or emulsified therein.

In preferred embodiments, the mousse, including each of Mousse 1-23, comprises a carrier that comprises, or consists essentially of, or consists of an organic solvent.

The present mousse, including each of Mousse 1-23, may comprise a carrier in the form of an oil-in-water emulsion or a water-in-oil emulsion.

Surface Tension Reducing Agent

In preferred embodiments the surface tension reducing agent comprises at least one surfactant. As appreciated by those skilled in the art, such surfactants not only provide a reduction in the surface tension of the carrier, preferably water, but may also act as an active component since in many preferred embodiments the surfactant also provides a cleansing effect to the formulation. The surfactant may also be present as emulsifier for emulsified active components (such as for example conditioning agents in the form of silicones and high molecular weight hydrocarbon materials) that may be present.

The surfactant suitable for use in the mousse of the present invention, including each of Mousse 1-23 can be anionic, cationic, non-ionic, amphoteric, or a combination of two or more of these.

Examples of anionic surfactants (including those that may also act as emulsifiers) suitable for use in the mousse of the present invention, including each of Mousse 1-23, include alkylarylsulphonates, e.g., sodium dodecylbenzene sulphonate, alkyl sulphates e.g., sodium lauryl sulphate, alkyl ether sulphates, e.g., sodium lauryl ether sulphate nEO, where n is from 1 to 20 alkylphenol ether sulphates, e.g., octylphenol ether sulphate nEO where n is from 1 to 20, sulphosuccinates, e.g., sodium dioctylsulphosuccinate, amino acid-based surfactants derived from natural coco fatty acid and the amino acid glycine, e.g. sodium cocoyl glycinate.

Examples of nonionic surfactants (including those that may also act as emulsifiers) suitable for use in the mousse of the present invention, including each of Mousse 1-23, are alkylphenol ethoxylates, e.g., nonylphenol ethoxylate nEO, where n is from 1 to 50, alcohol ethoxylates, e.g., lauryl alcohol nEO, where n is from 1 to 50, ester ethoxylates, e.g., polyoxyethylene monostearate where the number of oxyethylene units is from 1 to 30. Examples include: poly(oxy-1,2-ethanediyl), alpha-hydro-omega-hydroxy-, ether with D-glucitol (6:1), tetra-(9Z)-9-octadecenoate, which is also known as and referred to herein as Sorbeth-40 Tetraoleate: coco-glucoside and PEG-80 Sorbitan Laurate.

Amphoteric and zwitterionic cleansing surfactants suitable for use in the mousse of the present invention, including each of Mousse 1-23, may include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates and alkyl amidopropyl hydroxysultaines. Examples include lauryl amine oxide, cocodimethyl sulphopropyl betaine and preferably lauryl betaine, cocamidopropyl betaine and sodium cocamphopropionate, sodium lauroyl sarcosinate, sodium lauroamphocacetate.

Preferably the total amount of surfactant (including any used as emulsifier for another component of the formulation) is generally from 0.5% to about 50% based on the total weight of the mousse.

Preferably the total amount of surfactant (including any used as emulsifier for another component of the formulation) is generally from 3% to about 50% based on the total weight of the mousse.

Preferably the total amount of surfactant (including any used as emulsifier for another component of the formulation) is generally from 3% to about 25% based on the total weight of the mousse.

Preferably the total amount of surfactant (including any used as emulsifier for another component of the formulation) is generally from 10% to about 25% based on the total weight of the mousse.

Preferably the total amount of surfactant (including any used as emulsifier for another component of the formulation) is generally from 10% to about 20% based on the total weight of the mousse.

When the surfactant comprises, or consists essentially of, or consists of a surfactant selected from the group consisting of sodium cocoyl glycinate, cocamidopropyl betaine, sorbeth-40 tetraoleate, disodium cocaamphodiacetate and combinations of two or more of these, the total amount of surfactant (including any used as emulsifier for another component of the formulation) is preferably from 0.5% to less than 10% based on the total weight of the mousse, including each of Mousse 1-23.

When the surfactant comprises, or consists essentially of, or consists of a surfactant selected from the group consisting of sodium cocoyl glycinate, cocamidopropyl betaine, sorbeth-40 tetraoleate, disodium cocaamphodiacetate and combinations of two or more of these, the total amount of surfactant (including any used as emulsifier for another component of the formulation) is preferably from 0.5% to less than 5% based on the total weight of the mousse, including each of Mousse 1-23.

Preferably the mousse formulation of the present invention, including each of Mousse 1-23, comprises, or consists essentially of, or consists of a surfactant selected from the group consisting of sodium cocoyl glycinate, cocamidopropyl betaine, sorbeth-40 tetraoleate, disodium cocaamphodiacetate and combinations of two or more of these.

Foaming Agent

The foaming agent of the present invention acts to form from the carrier the foam form of the mousse, and in connection with the mousse methods, including each of Mousse Methods 1-23, and in connection with mousse dispensers, including each of Mousse Dispensers 1-23, also preferably acts to expel the concentrate from the container.

The mousse of the present invention preferably comprises foaming agent comprising, consisting essentially of or consisting of trans-1234ze.

The mousse of the present invention preferably comprises foaming agent comprising at least 30% by weight, based on the total foaming agent in the mousse, of trans-1234ze.

The mousse of the present invention preferably comprises foaming agent comprising at least 35% by weight, based on the total foaming agent in the mousse, of trans-1234ze.

The mousse of the present invention preferably comprises foaming agent comprising at least 40% by weight, based on the total foaming agent in the mousse, of trans-1234ze.

The mousse of the present invention preferably comprises foaming agent comprising at least 50% by weight, based on the total foaming agent in the mousse, of trans-1234ze.

The mousse of the present invention preferably comprises foaming agent comprising at least 60% by weight, based on the total foaming agent in the mousse, of trans-1234ze.

The mousse of the present invention preferably comprises foaming agent comprising at least 70% by weight, based on the total foaming agent in the mousse, of trans-1234ze.

The mousse of the present invention preferably comprises foaming agent comprising from about 30% to about 70% by weight of trans-1234ze, from about 4% to about 11% by weight of propane and from about 25% to about 60% by weight of isobutane.

The mousse of the present invention preferably comprises foaming agent comprising at least 80% by weight, based on the total foaming agent in the mousse, of trans-1234ze.

In preferred embodiments, from greater than 5% to about 20% by weight of the mousse is foaming agent.

Active Ingredients

The mousse of the present invention, including each of Mousse 1-23, includes in preferred embodiments one or more active components in addition to the surfactant.

In particular, such active components can be included by those skilled in the art in accordance with the intended functions of the Mousse.

The mousse of the present invention, including each of Mousse 1-23, includes, particularly in preferred hair styling mousse embodiments, active ingredients that impact the shape or texture of the hair.

The mousse of the present invention, including each of Mousse 1-23, includes, particularly in preferred hair conditioner mousse embodiments, hair conditioning components.

The mousse of the present invention, including each of Mousse 1-23, includes, particularly in preferred skin moisturizing mousse embodiments, skin moisturizers.

The mousse of the present invention, including each of Mousse 1-23, includes, particularly in preferred sun block mousse embodiments, components which protect the skin of the user from the sun.

The mousse of the present invention, including each of Mousse 1-23, includes, particularly in preferred hair color mousse embodiments, components which effect hair color.

Each of these active ingredients will be present in an amount effective to accomplish its purpose.

Adjuvants

The mousse of the present invention, including each of Mousse 1-23, includes in preferred embodiments one or more adjuvants that are included in the formulation in order to fine-tune one or more properties of the mousse to suit the needs for a particular mousse. In particular, such active components can be included by those skilled in the art in accordance with the intended functions of the mousse.

For example, the mousse of the present invention, including each of Mousse 1-23, may include for example one or more of: pigments or colouring agents to impact the color of the mousse; thickeners; film forming agents; pH adjusters; emulsifiers; rinse aids; fragrances; viscosity modifiers; preservatives; polyols such as glycerine and polypropylene glycol; and chelating agents such as EDTA. Each of these ingredients will be present in an amount effective to accomplish its purpose.

Methods

The present invention includes methods of forming a mousse having high gloss and good spreadability on a user, including each of Methods 1-23, where the mousse is a personal care mousse.

The present invention includes methods of forming a mousse having high gloss and good spreadability on a user, including each of Methods 1-23, where the mousse is a shampoo.

The present invention includes methods of forming a mousse having high gloss and good spreadability on a user, including each of Methods 1-23, where the mousse is a hand wash.

The present invention includes methods of forming a mousse having high gloss and good spreadability on a user, including each of Methods 1-23, where the mousse is a body wash.

The present invention includes methods of forming a mousse having high gloss and good spreadability on a user, including each of Methods 1-23, where the mousse is a facial cleanser.

The present invention includes methods of forming a mousse having high gloss and good spreadability on a user, including each of Methods 1-23, where the mousse is a skin conditioner.

The present invention includes methods of forming a mousse having high gloss and good spreadability on a user, including each of Methods 1-23, where the mousse is a perfume mousse.

The present invention includes methods of forming a mousse having high gloss and good spreadability on a user, including each of Methods 1-23, where the mousse is a skin moisturizer.

The present invention includes methods of forming a mousse having high gloss and good spreadability on a user, including each of Methods 1-23, where the mousse is a sun screen mousse.

The present invention includes methods of forming a mousse having high gloss and good spreadability on a user, including each of Methods 1-23, where the mousse is a leave-on facial mousse.

The present invention includes methods of forming a mousse having high gloss and good spreadability on a user, including each of Methods 1-23, where the mousse is a hair shaping mousse.

The present invention includes methods of forming a mousse having high gloss and good spreadability on a user, including each of Methods 1-23, where the mousse is a hair coloring mousse.

The present invention includes methods of forming a mousse having high gloss and good spreadability on a user, including each of Methods 1-23, where the mousse is a hair styling mousse.

The present invention includes methods of forming a mousse having high gloss and good spreadability on a user, including each of Methods 1-23, where the mousse is a hair conditioning mousse.

The present invention includes methods of forming a mousse having high gloss and good spreadability on a user, including each of Methods 1-23, where the mousse is a shave cream mousse.

The present invention includes methods of forming a mousse having high gloss and good spreadability on a user, including each of Methods 1-23, where the mousse is a cosmetic mousse.

The present invention includes methods of forming a mousse having high gloss and good spreadability on a user, including each of Methods 1-23, where the mousse is a medicinal/drug mousse.

The present invention includes methods of forming a mousse having high gloss and good spreadability on a user, including each of Methods 1-23, where the mousse is a cosmetic mousse and a medicinal/drug mousse.

The present invention includes methods of forming a mousse having high gloss and good spreadability on a user, including each of Methods 1-23, where the mousse is an antidandruff shampoo mousse.

The present invention includes methods of forming a mousse having high gloss and good spreadability on a user, including each of Methods 1-23, where the mousse is a sun tan mousse.

The present invention includes methods of forming a mousse having high gloss and good spreadability on a user, including each of Methods 1-23, where the mousse is a sun block mousse.

The present invention includes methods of forming a mousse having high gloss and good spreadability on a user, including each of Methods 1-23, where the mousse is an antiperspirant and/or deodorant mousse.

Dispensers

The present invention includes dispensers for dispensing a mousse having high gloss and good spreadability on a user, including each of Mousse Dispensers 1-23, where the mousse is a personal care mousse.

The present invention includes dispensers for dispensing a mousse having high gloss and good spreadability on a user, including each of Mousse Dispensers 1-23, where the mousse is a shampoo.

The present invention includes dispensers for dispensing a mousse having high gloss and good spreadability on a user, including each of Mousse Dispensers 1-23, where the mousse is a hand wash.

The present invention includes dispensers for dispensing a mousse having high gloss and good spreadability on a user, including each of Mousse Dispensers 1-23, where the mousse is a body wash.

The present invention includes dispensers for dispensing a mousse having high gloss and good spreadability on a user, including each of Mousse Dispensers 1-23, where the mousse is a skin moisturizer.

The present invention includes dispensers for dispensing a mousse having high gloss and good spreadability on a user, including each of Mousse Dispensers 1-23, where the mousse is a sun screen mousse.

The present invention includes dispensers for dispensing a mousse having high gloss and good spreadability on a user, including each of Mousse Dispensers 1-23, where the mousse is a leave-on facial mousse.

The present invention includes dispensers for dispensing a mousse having high gloss and good spreadability on a user, including each of Mousse Dispensers 1-23, where the mousse is a hair shaping mousse.

The present invention includes dispensers for dispensing a mousse having high gloss and good spreadability on a user, including each of Mousse Dispensers 1-23, where the mousse is a hair coloring mousse.

The present invention includes dispensers for dispensing a mousse having high gloss and good spreadability on a user, including each of Mousse Dispensers 1-23, where the mousse is a hair styling mousse.

The present invention includes dispensers for dispensing a mousse having high gloss and good spreadability on a user, including each of Mousse Dispensers 1-23, where the mousse is a hair conditioning mousse.

The present invention includes dispensers for dispensing a mousse having high gloss and good spreadability on a user, including each of Mousse Dispensers 1-23, where the mousse is a shave cream mousse.

The present invention includes dispensers for dispensing a mousse having high gloss and good spreadability on a user, including each of Mousse Dispensers 1-23, where the mousse is a cosmetic mousse.

The present invention includes dispensers for dispensing a mousse having high gloss and good spreadability on a user, including each of Mousse Dispensers 1-23, where the mousse is a medicinal/drug mousse.

The present invention includes dispensers for dispensing a mousse having high gloss and good spreadability on a user, including each of Mousse Dispensers 1-23, where the mousse is a cosmetic mousse and a medicinal/drug mousse.

The present invention includes dispensers for dispensing a mousse having high gloss and good spreadability on a user, including each of Mousse Dispensers 1-23, where the mousse is a antidandruff shampoo mousse.

The present invention includes dispensers for dispensing a mousse having high gloss and good spreadability on a user, including each of Mousse Dispensers 1-23, where the mousse is a shave cream mousse.

The present invention includes dispensers for dispensing a mousse having high gloss and good spreadability on a user, including each of Mousse Dispensers 1-23, where the mousse is a sun tan mousse.

The present invention includes dispensers for dispensing a mousse having high gloss and good spreadability on a user, including each of Mousse Dispensers 1-23, where the mousse is a sun block mousse.

The present invention includes dispensers for dispensing a mousse having high gloss and good spreadability on a user, including each of Mousse Dispensers 1-23, where the mousse is a facial makeup mousse.

The present invention includes dispensers for dispensing a mousse having high gloss and good spreadability on a user, including each of Mousse Dispensers 1-23, where the mousse is a deodorant mousse.

The present invention includes dispensers for dispensing a mousse having high gloss and good spreadability on a user, including each of Mousse Dispensers 1-23, where the mousse is an antiperspirant mousse.

### EXAMPLES

Comparative Examples C1A-C1C—Mousse Shampoo with 5%-10% A-46 Propellant

A mousse shampoo formulation was prepared using the ingredients and the amounts indicated in Comparative Table 1 below, with all amounts being in percentage by weight:

COMPARATIVE TABLE 1
Shampoo Mousse

| Component | | | Amount, | |
|---|---|---|---|---|
| No. | Component | Function | wt % | |
| 1 | Water | Carrier/Solvent | Qs to | |
| | | | 100 | |
| | Total Carrier (including pH adjust) | | | 78.15 |
| 2 | Disodium EDTA | Chelating agent | 0.05 | 0.05 |
| 3 | Glycerin | Humectant | 5 | 5 |
| 4 | Polyquaternium-10 | Hair conditioning | 0.3 | 0.3 |
| 5 | Sodium Lauroyl | Primary | 10 | |
| | Sarcosinate | Surfactant | | |
| 6 | Cocamidopropyl | Amphoteric | 3 | |
| | Betaine | Surfactant | | |
| 7 | Coco-Glucoside | Non-ionic | 2 | |
| | | surfactant | | |
| 8 | PEG-7 Glyceryl | Non-ionic | 1 | |
| | Cocoate | surfactant | | |
| | Total Surfactant | | 16 | |
| 9 | Citric Acid | Neutralizer | Adjust to | |
| | | | pH | |
| | | | ~6.0 | |
| 10 | DMDM Hydantoin | Preservatives | 0.5 | 0.5 |
| | TOTAL | | | 100 |

The formulation was formed by first adding the water into a mixing vessel. Then components 3-4 were added to the vessel sequentially in the order listed with mixing. Heat was then added while mixing to raise the temperature of the vessel contents to 70 C. Mixing was continued until a clear liquid phase was formed. The contents of the vessel were then cooled to room temperature (approximately 25C). Components 5-10 were then added sequentially in the order listed, mixing well between additions. The formulation was then charged into a can in an amount to produce a head-space in the can of about 20% based on the relative amount of the propellant to be added as described herein after. The can was then sealed. Propellant consisting of A-46 was then added to the can in an amount to produce can contents consisting of 95% by weight of the formulation of Comparative Table 1 and 5% by weight of A-46 propellant. This same experiment was then repeated but in successive experiments such that the A-46 was then added to the can in an amount to produce can contents consisting of 92.5% and 90% by weight of the formulation of Comparative Table 1 and 7.5% and 10% by weight of A-46 propellant, respectively.

The contents of the can from each experiment were then discharged by pressing the actuator for 1 second. The foam produced from each can was then evaluated and the results are reported in Table C1 below.

TABLE C1

| | A-46 AMOUNT | | |
|---|---|---|---|
| | | Example | Example |
| | Example C1A | C1B | C1C |
| Foam Property* | 5% | 7.5% | 10% |
| Initial Foam Stability | 4 | 5 | 5 |
| (scale from 1 (= foam | | | |
| not stable and flat) to 5 | | | |
| (foam very stable, | | | |
| puffy and volumized) | | | |
| Foam glossiness | 3 | 2 | 2 |
| (scale from 1 (= matte | | | |
| surface) to 5 (high | | | |
| gloss) | | | |
| Spread ability (scale | 4 | 3 | 2 |
| from 1 (= foam does | | | |
| not spread easily and | | | |
| form clumps) to 5 | | | |
| (spreads easily without | | | |
| clumps)) | | | |
| Foam density* | 0.069 | 0.038 | 0.030 |
| g/ml | | | |
| Foam Dispensing | 4 | 4 | 4 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by diving the measured weight by the volume of the can. A photograph of the resulting foam from each experiment is provided in Comparative Figures C1A, C1B and C1C.

As can be seen from the table and the Figures, this shampoo mousse formulation, which is representative of shampoo mousse formulations generally, when combined with from 5% to 10% by weight of A-46 as propellant produces a foam which has gloss properties that are disadvantageous for many important applications from a consumer appearance and use standpoint.

In addition, while the initial foam shape tends to improve with increased amount of A-46 propellant, increased A-46 propellant has a significant detrimental impact on the spreadability of the foam. In particular, the Example C1C foam was tested by dispensing onto the palm of the user (see Comparative FIG. C1D), and the user then attempted to spread the formulation onto the skin (see Comparative FIGS. C1E and C1F): As revealed by the photographs above, this formulation with 10% A-46 as the propellant performed very poorly from a spreadability standpoint. In particular, the foam did not spread evenly and resulted in large areas where the formulation was not present or only thinly present on the skin.

### Example 1A-1C—Mousse Shampoo with 5%, 7.5% and 10% trans-1234ze Propellant

The same mousse shampoo formulation as described in Comparative Example C1 was prepared, combined with propellant and dispensed as in Comparative Example 1, except that the propellant used consisted of trans-1234ze. The foam produced from each can was then evaluated and the results are reported in Table 1 below.

TABLE 1

| | trans-1234ze AMOUNT | | |
|---|---|---|---|
| | Example 1A | Example 1B | Example 1C |
| Foam Property* | 5% | 7.5% | 10% |
| Initial Foam Stability | 2 | 3 | 4 |
| (scale from 1 (= foam | | | |
| not stable and flat) to 5 | | | |
| (foam very stable, | | | |
| puffy and volumized) | | | |
| Foam glossiness | 5 | 5 | 4 |
| (scale from 1 (= matte | | | |
| surface) to 5 (high | | | |
| gloss) | | | |
| | 5 | 5 | 4 |
| Spread ability (scale | 5 | 5 | 5 |
| from 1 (= foam does | | | |
| not spread easily and | | | |
| form clumps) to 5 | | | |
| (spreads easily without | | | |
| clumps)) | | | |
| | 1 | 2 | 2 |
| Foam density | 0.148 | 0.118 | 0.068 |
| g/ml | | | |
| Foam Dispensing | 3 | 4 | 4 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by diving the measured weight by the volume of the can.

A photograph of the resulting foam from each experiment is provided in FIGS. 1A, 1B and 1C hereof.
As can be seen from the table and Figures, the gloss and wetness properties of the foam produced with trans-1234ze as the propellant produces foam having dramatically and unexpectedly superior gloss and wetness compared to the same foam formulation using A-46.

The foam from Example 1C (10% trans-1234ze as propellant) not only had the best initial foam stability performance, this foam also had excellent spreadability performance. In particular, the Example 1C foam was tested by dispensing onto the palm of the user (see FIG. 1D), and the user was able to easily and satisfactorily spread the formulation onto the skin (see FIGS. 1E and 1F).

Moreover, these examples illustrate applicants' discovery that unexpectedly advantageous foams can be produced when the amount of trans-1234ze used as the propellant is greater than 5%, more preferably from about 7.5% to about 10%. In particular, from the standpoint of initial foam stability, the foam produced using 5% trans-1234ze produces a foam quality that is below the mid-point on this scale. In particular, the foam made with 5% trans-1234ze is relatively flat and contains surface holes and bubbles which would result it in being considered of poor quantity and shape from a consumer appearance and use standpoint compared to a puffier foam with no visible holes or surface bubbles. On the other hand, as can be seen from the Photo 1B above, the use of 7.5% by weight of trans-1234ze as propellant produces a foam of a greatly and unexpectedly improved stability relative to foam produced in Example 1A. In particular, the foam produced by Example 1B with 7.5% of trans-1234ze is higher (i.e., not as flat) and has fewer surface holes and bubbles and as such would be considered of a superior quantity and shape from a consumer appearance and use standpoint than the foam of Example 1A. Further, as can be seen from the FIG. 1C, the use of 10% by weight of trans-1234zeas a propellant produces a foam of a greatly and unexpectedly improved initial stability relative to foam produced in Example 1A. In particular, the foam produced by Example 1C with 10% of trans-1234ze is higher and more dome-shaped (i.e., not as flat) as the foams produced with 5% and 7.5% of trans-1234ze and exhibits essentially no visible surface holes or bubbles and as such would be considered of excellent quantity and shape from a consumer appearance and use standpoint.

### Comparative Examples C2A-C2C—Shaving Mousse with 5%-10% A-46 Propellant

A shaving mousse formulation was prepared using the ingredients and amounts indicated in Comparative Table 2 below:

COMPARATIVE TABLE 2
Shaving Mousse

| Component | | | Amount, | |
|---|---|---|---|---|
| No. | Component | Function | wt % | |
| 1 | Water | Carrier/Solvent | Qs to 100 | |
| 2 | Triethanolamine | Neutralizer | 3.5 | |
| | Total Carrier (including neutralizer) | | | 91.45 |
| 3 | Stearic Acid | Surface tension | 5 | |
| | | reducer (and fatty | | |
| | | acid soap) | | |
| 4 | Myristic Acid | Surface tension | 2 | |
| | | reducer (and fatty | | |
| | | acid soap) | | |
| | Total Surface Tension Reducer | | | 7 |
| 5 | Disodium EDTA | Chelating agent | 0.05 | 0.05 |
| 6 | PVP-K30 (PVP) | Film Former | 1 | 1 |
| 7 | Stearic Acid | Surface tension | 5 | 5 |
| | | reducer (and fatty | | |
| | | acid soap) | | |
| 8 | Propylene Glycol | humectant | 3 | 3 |
| 9 | Lanolin | occlusive | 2 | 2 |
| 10 | Mineral oil | Emollient | 1 | 1 |
| 11 | DMDM Hydantoin | Preservatives | 0.5 | 0.5 |
| | TOTAL | | | 100 |

The formulation was formed by adding components 1, 2 and 3 into a first vessel and by adding components 4-10 into a second vessel. The contents of each vessel were then heated to a temperature of from about 70° C. to about 75° C. The contents of the first vessel were then added to the contents of the second vessel with stirring. The formulation was then charged into a can in an amount to produce a head-space in the can of about 20% based on the relative amount of the propellant to be added as described herein after. The can was then sealed. Propellant consisting of A-46 was then added to the can in an amount to produce can contents consisting of 95% by weight of the formulation of Comparative Table 2 and 5% by weight of A-46 propellant (Example C2A). This same experiment was then repeated, except that in Experiment C2B and C2C, A-46 was added to the can in an amount to produce can contents consisting of 92.5% and 90% by weight of the formulation of Comparative Table 2 and 7.5% and 10% by weight of A-46 propellant, respectively.

The foam produced from each can was then evaluated and the results are reported in Table C2 below.

TABLE C2

| | A-46 AMOUNT | | |
|---|---|---|---|
| | Example | Example | Example |
| | C2A | C2B | C2C |
| Foam Property* | 5% | 7.5% | 10% |
| Initial Foam Stability | 4 | 4 | 3 |
| (scale from 1 (= foam | | | |
| not stable and flat) to 5 | | | |
| (foam very stable, | | | |
| puffy and volumized) | | | |
| Foam glossiness | 2 | 1 | 1 |
| (scale from 1 (= matte | | | |
| surface) to 5 (high | | | |
| gloss) | | | |
| Spread ability (scale | 4 | 2 | 1 |
| from 1 (= foam does | | | |
| not spread easily and | | | |
| form clumps) to 5 | | | |
| (spreads easily without | | | |
| clumps)) | | | |
| Foam density* | 0.066 | 0.036 | 0.026 |
| g/ml | | | |
| Foam Dispensing | 4 | 2 | 1 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by dividing the measured weight by the volume of the can. A photograph of the resulting foam from each experiment is provided in Figures C2A, C2B and C2C hereof.

As can be seen from the table and photographs above, this shaving mousse formulation, which is representative of shaving mousse formulations generally, when combined with from 5% to 10% by weight of A-46 as propellant produces a foam which has gloss, wetness and sprayability properties that are disadvantageous for many important applications from a consumer appearance and use standpoint.

### Example 2A-2C—Shaving Mousse with 5%, 7.5% and 10% trans-1234ze Propellant

The same shaving mousse formulation as described in Comparative Example C2 was prepared, combined with propellant and dispensed as in Comparative Example 2, except that the propellant used consisted of trans-1234ze. The foam produced from each can was then evaluated and the results are reported in Table 2 below.

TABLE 2

| | trans-1234ze AMOUNT | | |
|---|---|---|---|
| | Example 2A | Example 2B | Example 2C |
| Foam Property* | 5% | 7.5% | 10% |
| Initial Foam Stability | 3 | 5 | 4 |
| (scale from 1 (= foam | | | |
| not stable and flat) to 5 | | | |
| (foam very stable, | | | |
| puffy and volumized) | | | |
| Foam glossiness | 5 | 3 | 2 |
| (scale from 1 (= matte | | | |
| surface) to 5 (high | | | |
| gloss) | | | |
| Spread ability (scale | 5 | 5 | 4 |
| from 1 (= foam does | | | |
| not spread easily and | | | |
| form clumps) to 5 | | | |
| (spreads easily without | | | |
| clumps)) | | | |
| Foam density* | 0.237 | 0.147 | 0.132 |
| g/ml | | | |
| Foam Dispensing | 3 | 5 | 4 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by diving the measured weight by the volume of the can.

A photograph of the resulting foam from each experiment is provided in FIGS. 2A, 2B and 2C hereof.

As can be seen from the table and photographs above, the glossiness, wetness, spreadability and sprayability properties of the foam produced with trans-1234ze as the propellant are dramatically and unexpectedly superior to the properties of the same foam formulation but using A-46 as the propellant. Moreover, these examples illustrate applicants' discovery that unexpectedly advantageous foams can be produced when the amount of trans-1234ze used as the propellant is greater than 5%, more preferably from about 7.5% to about 10 In particular, from the standpoint of foam shape and quantity, the foam produced using 5% trans-1234ze produces a foam quality that is near or below the mid-point on each of these scales. In particular, the foam made with 5% trans-1234ze is relatively flat and has numerous relatively larger (readily visible) surface holes and bubbles. This combination of conditions would result in the foam being considered of relatively poor quantity and shape from a consumer appearance and use standpoint compared to a higher foam with no visible holes or surface bubbles. On the other hand, as can be seen from the FIG. 2B, the use of 7.5% by weight of trans-1234ze as propellant produces a foam of a greatly and unexpectedly improved quantity and shape relative to foam produced in Example 2A. In particular, the foam produced by Example 2B with 7.5% of trans-1234ze is higher (i.e., not as flat and more dome-shaped) and has fewer surface holes and bubbles and as such would be considered of a superior quantity and shape from a consumer appearance and use standpoint than the foam of Example 2A. Further, as can be seen from the FIG. 2C above, the use of 10% by weight of trans-1234ze as propellant produces a foam of a greatly and unexpectedly improved quantity and shape relative to foam produced in Example 2A. In particular, the foam produced by Example 2C with 10% of trans-1234ze is higher (i.e., not as flat and more dome-shaped) than Examples 2A and 2B and exhibits essentially no visible surface holes or bubbles and as such would be considered of excellent quantity and shape from a consumer appearance and use standpoint.

### Comparative Examples C3A-C3C—Styling Mousse with 5%-10% A-46 Propellant

A styling mousse formulation was prepared using the ingredients and amounts indicated in Comparative Table 3 below:

Comparative TABLE 3
Styling Mousse

| Component | | | Amount, | |
|---|---|---|---|---|
| No. | Component | Function | wt % | |
| 1 | Water | Carrier/Solvent | Qs to | |
| | | | 100 | |
| | Total Carrier (including neutralizer) | | | 95.6 |
| 2 | Polyquaternium-69 in water | Surface tension | 2.25 | |
| | | reducer (and | | |
| | | film former) | | |
| 3 | Palmitamidopropyltrimonium | Surface tension | 0.65 | |
| | Chloride | reducer (and | | |
| | | emulsifier) | | |
| | Total Surface Tension Reducer | | | 2.9 |
| 4 | VP/Dimethylaminoethylmethacrylate | film former | 1 | 1 |
| | copolymer | | | |
| 5 | DMDM Hydantoin | Preservatives | 0.5 | 0.5 |
| | TOTAL | | | 100 |

The formulation was formed by adding the components to a vessel in the order listed with stirring with each addition. A clear liquid is formed. The formulation was then charged into a can in an amount to produce a head-space in the can of about 20% based on the relative amount of the propellant to be added as described herein after. The can was then sealed. Propellant consisting of A-46 was then added to the can in an amount to produce can contents consisting of 95% by weight of the formulation of Comparative Table 3 and 5% by weight of A-46 propellant (Example C3A). This same experiment was then repeated except that in Experiment C3B and C3C, A-46 was added to the can in an amount to produce can contents consisting of 92.5% and 90% by weight of the formulation of Comparative Table 3 and 7.5% and 10% by weight of A-46 propellant, respectively.

The foam produced from each can was then evaluated and the results are reported in Table C3 below.

TABLE C3

| | A-46 AMOUNT | | |
|---|---|---|---|
| | Example | Example | Example |
| | C3A | C3B | C3C |
| Foam Property* | 5% | 7.5% | 10% |
| Initial Foam Stability | 3 | 4 | 4 |
| (scale from 1 (= foam | | | |
| not stable and flat) to 5 | | | |
| (foam very stable, | | | |
| puffy and volumized) | | | |
| Foam glossiness | 3 | 3 | 3 |
| (scale from 1 (= matte | | | |
| surface) to 5 (high | | | |
| gloss) | | | |
| Spread ability (scale | 4 | 2 | 1 |
| from 1 (= foam does | | | |
| not spread easily and | | | |
| form clumps) to 5 | | | |
| (spreads easily without | | | |
| clumps)) | | | |
| Foam density* | 0.053 | 0.035 | 0.013 |
| g/ml | | | |
| Foam Dispensing | 2 | 3 | 3 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by diving the measured weight by the volume of the can.

A photograph of the resulting foam from each experiment is provided in FIGS. C3A, C3B and C3C hereof.

As can be seen from the table and photographs above, this shaving mousse formulation, which is representative of styling mousse formulations generally, when combined with from 5% to 10% by weight of A-46 as propellant produces a foam which has glossiness, wetness, spreadability and sprayability that are disadvantageous for many important applications from a consumer appearance and use standpoint.

### Example 3A-3C—Styling Mousse with 5%, 7.5% and 10% trans-1234ze Propellant

The same styling mousse formulation as described in Comparative Example C3 was prepared, combined with propellant and dispensed as in Comparative Example 3, except that the propellant used consisted of trans-1234ze. The foam produced from each can was then evaluated and the results are reported in Table 3 below.

TABLE 3

| | trans-1234ze AMOUNT | | |
|---|---|---|---|
| | Example 3A | Example 3B | Example 3C |
| Foam Property* | 5% | 7.5% | 10% |
| Initial Foam Stability | 3 | 4 | 5 |
| (scale from 1 (= foam | | | |
| not stable and flat) to 5 | | | |
| (foam very stable, | | | |
| puffy and volumized) | | | |
| Foam glossiness | 5 | 4 | 4 |
| (scale from 1 (= matte | | | |
| surface) to 5 (high | | | |
| gloss) | | | |
| Spread ability (scale | 5 | 5 | 4 |
| from 1 (= foam does | | | |
| not spread easily and | | | |
| form clumps) to 5 | | | |
| (spreads easily without | | | |
| clumps)) | | | |
| Foam density* | 0.150 | 0.087 | 0.054 |
| g/ml | | | |
| Foam Dispensing | 4 | 4 | 5 |
| (scale from 1 (= | | | |
| blasting spray or weak | | | |
| liquid dispersion) to 5 | | | |
| (dispensing well | | | |
| without problems)) | | | |

*The density properties reported herein are measured by filling a 25 mL can to the rim with the foam and measuring the weight of the foam. Density is calculated by dividing the measured weight by the volume of the can.

A photograph of the resulting foam from each experiment is provided in FIGS. 3A, 3B and 3C hereof.

As can be seen from the table and Figures, the glossiness, wetness, spreadability and sprayability properties of the foam produced with trans-1234ze as the propellant are dramatically and unexpectedly superior to the properties of the same foam formulation but using A-46 as the propellant. Moreover, these examples illustrate applicants' discovery that unexpectedly advantageous foams can be produced when the amount of trans-1234ze used as the propellant is greater than 5%, more preferably from about 7.5% to about 10%. In particular, from the standpoint of foam shape, stability and quantity, the foam produced using 5% trans-1234ze produces a foam quality that is near or below the mid-point on each of these scales. In particular, the foam made with 5% trans-1234ze is relatively flat and has numerous relatively larger (readily visible) surface holes and bubbles, and the stability is 2. This combination of conditions would result in the foam being considered of relatively poor quantity, stability and shape from a consumer appearance and use standpoint compared to a higher foam with no visible holes or surface bubbles. On the other hand, as can be seen from the FIG. 3B above, the use of 7.5% by weight of trans-1234ze as propellant produces a foam of a greatly and unexpectedly improved shape, stability and quantity relative to foam produced in Example 3A. In particular, the foam produced by Example 3B with 7.5% of trans-1234ze is higher and more dome-shaped (i.e., not as flat) and has fewer surface holes and bubbles and as such would be considered of a superior quantity and shape from a consumer appearance and use standpoint than the foam of Example 3A. Further, as can be seen from the FIG. 3C above, the use of 10% by weight of trans-1234ze as propellant produces a foam of a greatly and unexpectedly improved quantity and shape relative to foam produced in Example 3A. In particular, the foam produced by Example 3C with 10% of trans-1234ze is yet higher and more dome-shaped (i.e., not as flat) and exhibits essentially no visible surface holes or bubbles and as such would be considered of excellent quantity and shape from a consumer appearance and use standpoint.

### Example 4A-4I—Mousse Shampoo with Propellant Comprising trans-1234ze and A-46

The same shampoo mousse formulation as described in Comparative Example C1 is used with a series of propellants comprising trans-1234ze and the propellant A-46. The different propellant blends tested are described in Table 4 below:

TABLE 4

| | | Wt % | | Wt % | Wt % | Wt % |
|---|---|---|---|---|---|---|
| Ex. | Blend | trans- | Wt % | trans- | pro- | iso- |
| No. | No. | 1234ze | A-46 | 1234ze | pane | butane |
| 4A | 1 | 90 | 10 | 90 | 1.52 | 8.48 |
| 4B | 2 | 80 | 20 | 80 | 3 | 17 |
| 4C | 3 | 70 | 30 | 70 | 4.5 | 25.5 |
| 4D | 4 | 60 | 40 | 60 | 6.1 | 33.9 |
| 4E | 5 | 50 | 50 | 50 | 7.6 | 42.4 |
| 4F | 6 | 40 | 60 | 40 | 9.1 | 50.9 |
| 4J | 7 | 30 | 70 | 30 | 10.6 | 59.4 |
| 4H | 8 | 20 | 80 | 20 | 12.1 | 67.9 |
| 4I | 9 | 10 | 90 | 10 | 13.6 | 76.4 |

A can containing 90% weight by mousse formulation and 10% of each of Propellants 1-9 is prepared and the foam is dispensed from each of the 9 cans onto a horizontal sheet of black craft paper and allowed to form for 3 minutes after dispensing is shown side-by-side in FIG. 4A hereof.
This experiment reveals that an unexpected advantage is achieved using for mousse formulations with propellant comprising a combination of trans-1234ze and A-46 in a trans-1234ze:A-46 weight ratio of from about 7:3 to about 3:7, and even more preferably from about 7:3 to about 4:6, with ratios of from about 7:3 to about 5:5 being highly preferred. In particular, these results show that formulations using the preferred ratios of trans-1234ze and A-46 in the blend provide foams having unexpectedly high levels of performance in foam shape, glossiness, wetness, stability and quantity.

In addition, a can containing 90% weight by mousse formulation and 10% of each of Propellants 1-9 is prepared and foam is dispensed from each of the 9 cans onto a vertical sheet of black craft paper, and allowed to form for 3 minutes after dispensing is shown side-by-side in FIG. 4B hereof:

This experiment reveals that an unexpected advantage in terms of excellent foam spreadability and stability is achieved with propellant comprising a combination of trans-1234ze and A-46 in a trans-1234ze:A-46 weight ratio of from about 6:4 to about 4:6, and even more preferably from about 6:4 to about 5:5.

### Comparative Example C5—Sunscreen Mousse 10% A-46 Propellant

A sunscreen mousse was prepared using the ingredients and the amounts indicated in Comparative Table 5 below, with all amounts being in percentage by weight:

TABLE C5
Sunscreen Mousse

| Component | | | Amount, | |
|---|---|---|---|---|
| No. | Component | Function | wt % | |
| 1 | Water | Carrier/Solvent | Qs to | |
| | | | 100 | |
| | Total Carrier (including neutralizer) | | | 59.25 |
| 2 | Disodium EDTA | Chelating agent | .05 | 0.05 |
| 3 | Natrosol 250 HX | Thickner | .2 | 0.2 |
| | (Hydroxyethyl cellulose) | | | |
| 4 | Arlacel 165 (PEG-100 | Surface tension | 1.2 | |
| | Stearate and Glyceryl | reducer (and | | |
| | Stearate) | emulsifier) | | |
| 5 | Tego Alkanol IC (lsoceteth-20) | Surface tension | 1 | |
| | | reducer (and | | |
| | | emulsifier) | | |
| 6 | Polysorbate-20 | Surface tension | 1 | |
| | | reducer (and | | |
| | | emulsifier) | | |
| | Total Surface Tension Reducer | | | 3.2 |
| 7 | Caprylic Capric Triglyceride | Emollient | 4 | 4 |
| 8 | Propylene Glycol | humectant | 3 | 3 |
| 9 | C12-15 Alkyl Benzoate | emollient | 4 | 4 |
| 10 | Ganex V-220 (PVP (and) | film former | 1 | 1 |
| | VP/Eicosene Copolymer) | | | |
| 11 | Octocrylene | sunfilter | 5 | 5 |
| 12 | Homosalate | sunfilter | 7 | 7 |
| 13 | Octisalatae | sunfilter | 8 | 8 |
| 14 | Avobenzone | sunfilter | 2 | 2 |
| 15 | Beeswax | Wax | 0.3 | 0.3 |
| 16 | Dry Flo TS (Tapioca Starch | Aesthetic Modifier | 1 | 1 |
| | (and) | | | |
| | Polymethylsilsesquioxane) | | | |
| 17 | Tapioca Pure (Tapioca | Aesthetic Modifier | 1 | 1 |
| | Starch) | | | |
| 18 | Chlorphenesin | Preservative | 0.2 | 0.2 |
| 19 | Euxyl PE9010 | Preservative | 0.8 | 0.8 |
| | TOTAL | | | 100 |

The formulation was formed by first preparing an oil phase comprising the sunfilters, beeswax, Tego alkanol IC20, Arlacel 165, ganex v220, c12-15 alkyl benzoate, caprylic capric triglyceride at a temperature of 70 C—80 C and mix those components until uniform. A water phase is then formed by adding into a separate container the water and then dispersing the Natrosol 250 HX until uniform. The water phase is then heated to 70 C-75 C, and the following components are then added: disodium EDTA, chlorphenesin, propylene glycol, and polysorbate-20. The water phase components are then mixed until uniform. Emulsification is achieved by adding the oil phase to the water phase with homogenization until opaque emulsion formed.

The formulation was then charged into a can in an amount to produce a head-space in the can of about 20% based on the relative amount of the propellant to be added as described herein after. The can was then sealed. Propellant consisting of A-46 was then added to the can in an amount to produce can contents consisting of 90% by weight of the formulation of Table C5 and 10% by weight of A-46 (Example C5A).

In addition, foam made with higher levels of A-46 propellant has a significant detrimental impact on the spreadability of the foam. In particular, the foam was tested by dispensing onto the back of the hand of the user (see FIG. C5A), and the user then attempted to spread the formulation onto the skin (see FIGS. C5B and C5C).

As revealed by FIGS. C5A, C5B and C5C, this formulation with 10% A-46 as the propellant performed very poorly from a spreadability standpoint. In particular, the foam did not spread evenly and resulted in large clumped areas and left large areas where the formulation was not present on the skin.

### Example 5—Sunscreen Mousse with 10% trans-1234ze Propellant

A sunscreen mousse formulation was prepared as described in Comparative Example 5 and combined with propellant and dispensed as in Comparative Example 5, except that the propellant used consisted of trans-1234ze such that the can had 10% by weight of trans-1234ze. This foam was dispensed onto the back of the hand of a user (see FIG. 5A), and the user was able to easily and satisfactorily spread the formulation onto the skin (see FIGS. 5B and 5C).

### Example 6—Mousse Skin Cleanser with Propellant Comprising trans-1234ze and Different Surfactants

A mousse skin formulation was used to prepare and compare a series of mousse foams made using trans-1234ze as the propellant in an amount of about 10 wt % of the formulation contents and a series of different surfactants, with the balance being water. A can containing the mousse formulation with 2.5 wt %, 5 wt % and 10 wt % of the tested surfactant is prepared and the foam is dispensed from each of the cans and the foam density is measured. The results are reported in Table 6A below and in FIG. 6A (with the first bar for each surfactant being at 2.5%, the second bar being at 5% and the third bar being at 10%).

TABLE 6A

| Surfactant | | | | |
|---|---|---|---|---|
| type | Surfactant | 10 wt % | 5 wt % | 2.5 wt % |
| Anionic | Sodium Laureth Sulfate | 0.063 | 0.076 | 0.153 |
| SAA | Sodium Cocoyl Glycinate | 0.058 | 0.048 | 0.040 |
| | Sodium Cocoyl Alaninate | 0.069 | 0.082 | 0.093 |
| | Sodium Cocoyl Glutamate | 0.057 | 0.100 | 0.150 |
| | Sodium Methyl Cocoyl Taurate | 0.056 | 0.072 | 0.123 |
| | Sodium Lauroyl Sarcosinate | 0.086 | 0.162 | 0.220 |
| Amphoteric | Cocamidopropyl Betaine | 0.086 | 0.075 | 0.053 |
| SAA | Sodium Lauroamphoacetate | 0.051 | 0.091 | 0.067 |
| | Disodium cocoamphodiacetate | 0.063 | 0.079 | 0.057 |
| nonionic | PEG-80 Sorbitan Laurate | 0.085 | 0.123 | 0.100 |
| SAA | Coco-Glucoside | 0.061 | 0.068 | 0.073 |
| | Sorbeth-40 Tetraoleate | 0.122 | 0.100 | 0.183 |

As can be seen from the data above, generally the foam density of the mousse increases as the concentration of surfactant decreases. However, applicants have unexpectedly found that a small limited number of surfactants when used in the mousse formulations of the present invention, including each of Mousse 1-23, provide a unexpected opposite result, that is, the foam density decreases as surfactant concentration decreases. This result is illustrated in FIG. 6B hereof, which shows the foam volume produced (which is inversely related to foam density) for the same surfactants as reported in Table 6A for surfactant concentrations of 2.5 wt %, 5 wt % and 10 wt %.

1. A mousse composition having high gloss and good spreadability comprising:
(i) from about 50% by weight to about 90% by weight, based on the weight of components (i)-(v), of a carrier;
(ii) from about 0.5% to about 20% by weight, based on the weight of components (i)-(v), of one or more surface tension reducing agents that reduce the surface tension of the carrier;
(iii) from greater than 5% to about 20% by weight, based on the weight of components (i)-(v), of a foaming agent, wherein said foaming agent comprises at least about 30% by weight of trans-1234ze; and
(iv) optionally one or more active components in addition to said surface tension reducing agents;
(v) optionally one or more adjuvants.

2. The mousse composition of claim 1, wherein said carrier comprises at least 90% by weight of water.

3. The mousse composition of claim 1, wherein said foaming agent comprises at least about 40% by weight of trans-1234ze.

4. The mousse composition of claim 1, comprising from about 6% to about 20% by weight, based on the weight of components (i)-(v) of one or more surface tension reducing agents that reduce the surface tension of the carrier and from about 7.5% to about 15% by weight, based on the weight of components (i)-(v), of foaming agent.

5. The mousse composition of claim 1, wherein said foaming agent comprises from about 30% to about 70% by weight of trans-1234ze, from about 4% to about 11% by weight of propane and from about 25% to about 60% by weight of isobutene.

6. The mousse composition of claim 1, wherein said foam has a spreadability of 4 or greater, a glossiness of 4 or greater, an initial foam stability of 3 or greater.

7. The mousse composition of claim 1, provided that if said surface tension modifier is present in an amount of less than about 3% by weight, then said component (v) is present and comprises an adjuvant which aids in the formation of foam cell walls from said carrier.

8. A method of treating a human comprising applying a mousse according to claim 1 to the skin and/or hair of the human.

9. The method of claim 8, wherein a surface tension reducing agent is selected from a group consisting of sodium cocoyl glycinate, cocamidopropyl betaine, sorbeth-40 tetraoleate, disodium cocaamphodiacetate and combinations of two or more of these, and wherein said method of applying comprises applying said mousse formulation to the user to form a mousse foam having a density of about 0.05 g/ml or less.

10. An article of manufacture comprising a container under pressure and a mousse according to claim 1 in the container.
